(19) United States
(12) Patent Application Publication
(10) Pub. No.: US 2026/0187984 A1
(43) Pub. Date: Jul. 2, 2026

(54) LESION-AWARE CHEST X-RAY SYNTHESIS FOR IMPROVING THORACIC DISEASE DETECTION

(71) Applicant: The Hong Kong University of Science and Technology, Hong Kong (CN)

(72) Inventors: Hao CHEN, Hong Kong (CN); Haibo JIN, Hong Kong (CN)

(21) Appl. No.: 19/129,775

(22) PCT Filed: Jul. 18, 2023
(86) PCT No.: PCT/CN2023/107874

§ 371 Date: May 14, 2025

(51) Int. Cl. G06V 10/774; G06N 3/094; G06T 7/00; G16H 30/40

(52) U.S. Cl. CPC G06V 10/774; G06T 7/0012; G16H 30/40; G06N 3/094; G06T 2207/20081; G06T 2207/20084; G06T 2207/30096

(57) ABSTRACT

Techniques are described for augmenting chest X-ray (CXR) images with synthetic lesions using a lesion-aware machine learning framework in association with optimizing thoracic disease detection models. In an example, a system can comprise a lesion augmentation component that generates synthetic lesion images comprising synthetic lesion image data objects integrated on or within medical images using lesion generator, trained to generate the synthetic lesion image data objects, and tailor the synthetic lesion image data objects to account for different types of lesions and different anatomical locations of the lesions. The system can further comprise a training component that trains a lesion detector using the synthetic lesion images. The training component can further perform an alternate training strategy to integrate the training of the lesion generator and the detector for mutual boosting.

### TECHNICAL FIELD

This application relates to techniques for augmenting chest X-ray (CXR) images with synthetic lesions using a lesion-aware machine learning framework in association with optimizing thoracic disease detection models.

### BACKGROUND

Chest X-ray (CXR) is the most common exam for screening thoracic diseases due to its advantage in cost-effectiveness and low-dose radiation. The increasing number of CXR exams brings heavy workload to radiologists. Moreover, CXR interpretation can be challenging even for experienced radiologists due to the complexity of chest anatomy and the subtle variations in lesion areas.

With the development of deep learning techniques, great progress has been made in computer-aided diagnosis (CAD) of thoracic diseases, which is promising in easing the burden of radiologists. However, training a robust model for disease diagnosis and lesion localization requires a large amount of samples with fine-grained labels, which is difficult to obtain due to privacy issues and expensive labeling cost.

### SUMMARY

The following presents a summary to provide a basic understanding of one or more embodiments of the invention. This summary is not intended to identify key or critical elements or delineate any scope of the different embodiments or any scope of the claims. Its sole purpose is to present concepts in a simplified form as a prelude to the more detailed description that is presented later. In one or more embodiments, systems, computer-implemented methods, apparatus and/or computer program products are described that facilitate augmenting chest X-ray (CXR) images with synthetic lesions using a lesion-aware machine learning framework in association with optimizing thoracic disease detection models. In some embodiments, the disclosed techniques can be applied to other types of lesions associated with other types of diseases and anatomical regions of the body. The disclosed techniques can also be extended to other medical imaging modalities (e.g., magnetic resonance imaging (MRI), computed tomography (CT), and others).

According to an embodiment, a system is provided that comprises a memory that stores computer-executable components, and a processor that executes the computer-executable components stored in the memory. The computer-executable components can comprise a lesion augmentation component that generates synthetic lesion images comprising synthetic lesion image data objects integrated on or within medical images using a synthetic lesion generation model, wherein the model is trained to generate the synthetic lesion image data objects and tailor the synthetic lesion image data objects to account for different types of lesions and different anatomical locations of the lesions. In one or more embodiments, the different types of lesions correspond to different types of thoracic diseases and/or lesions associated with the different types of thoracic diseases (e.g., a mass, a nodule, a pneumonia lesion, a tuberculosis lesion, a fracture, etc.). In various embodiments, the medical images comprise CXR images.

In one or more embodiments, the lesion augmentation component adds the synthetic lesion images to a lesion image training dataset comprising lesion images, the lesion images comprising the augmented medial images, and wherein the computer-executable components further comprise a training component that employs the lesion image training dataset to train a lesion detection model to detect the different types of lesions in the lesion images. In various embodiments, the lesion augmentation component generates the synthetic lesion images in association with reception of annotation data indicating a defined disease type, a defined anatomical location and a defined size of respective objects of the synthetic lesion image data objects for integration on or within the medical images, and wherein the training component employs the annotation data respectively associated with the synthetic lesion images as ground truth (GT) information in association with training the lesion detection model. The computer-executable components can further comprise a performance assessment component that identifies one or more target lesion images of the lesion image training dataset associated with a negative performance criterion of the lesion detection model, and wherein the training component updates the synthetic lesion generation model based on the one or more target lesion images.

In this regard, the training component can also train the synthetic lesion generation model using one or more machine learning processes to tailor the synthetic lesion data objects to account for different types of lesions and anatomical locations of the lesions. In some embodiments, the synthetic lesion generation model can also tailor the synthetic lesions to account for different sizes and textures of the lesions. In some embodiments, the one or more machine learning processes comprise an adversarial training process employing a lesion generator network and a discriminator network, wherein the lesion generator network comprises convolutional layers and transformers. In some implementations, the synthetic lesion generation model can comprise a style variation module that generates different style variations of the synthetic lesion image data objects using noise injection.

In some embodiments, elements described in connection with the disclosed systems can be embodied in different forms such as a computer-implemented method, a computer program product, or another form.

### DESCRIPTION OF THE DRAWINGS

FIG. 1 presents an example, non-limiting computing system that facilitates augmenting CXR images with synthetic lesions and employing the augmented images in association with optimizing a thoracic disease detection model, in accordance with one or more embodiments of the disclosed subject matter.

FIG. 2 presents example CXRs with different types of lesions associated with different types of thoracic diseases.

FIG. 3 presents an example process for training a synthetic lesion generation model in accordance with one or more embodiments of the disclosed subject matter.

FIG. 4 presents an example synthetic lesion generation model in accordance with one or more embodiments of the disclosed subject matter.

FIG. 5 illustrates example components of an example synthetic lesion generation model in accordance with one or more embodiments of the disclosed subject matter.

FIGS. 6A and 6B present a table illustrating example synthetic lesion images generated by a synthetic lesion generation model in accordance with one or more embodiments of the disclosed subject matter.

FIG. 7 presents examples of different types of styles of synthetic lesion objects capable of being generated by the synthetic lesion generation model via the style variation component in accordance with one or more embodiments of the disclosed subject matter.

FIGS. 8A and 8B present a flow diagram of an example process for training the lesion detector and the lesion generator in accordance with one or more embodiments of the disclosed subject matter.

FIGS. 9A and 9B present a flow diagram of another example process for training the lesion detector and the lesion generator in accordance with one or more embodiments of the disclosed subject matter.

FIG. 10 presents a high-level illustration of an alternate training framework for training the lesion generator and the lesion detector for mutual boosting in accordance with one or more embodiments of the disclosed subject matter.

FIG. 11 illustrates a block diagram of an example, non-limiting computer implemented method for augmenting CXR images with synthetic lesions in accordance with one or more embodiments of the disclosed subject matter.

FIG. 12 illustrates a block diagram of another example, non-limiting computer implemented method for augmenting CXR images with synthetic lesions in accordance with one or more embodiments of the disclosed subject matter.

FIG. 13 illustrates a block diagram of an example, non-limiting computer implemented method for augmenting CXR images with synthetic lesions and employing the augmented images in association with optimizing a thoracic disease detection model, in accordance with one or more embodiments of the disclosed subject matter.

FIG. 14 illustrates a block diagram of an example, non-limiting operating environment in which one or more embodiments described herein can be facilitated.

FIG. 15 illustrates a block diagram of another example, non-limiting operating environment in which one or more embodiments described herein can be facilitated.

### DETAILED DESCRIPTION

The following detailed description is merely illustrative and is not intended to limit embodiments and/or application or uses of embodiments. Furthermore, there is no intention to be bound by any expressed or implied information presented in the preceding Background section, Summary section or in the Detailed Description section.

The subject disclosure provides systems, computer-implemented methods, apparatus and/or computer program products are described that facilitate augmenting chest X-ray (CXR) images with synthetic lesions using a lesion-aware machine learning (ML) framework in association with optimizing thoracic disease detection models.

As noted in the Background Section, training a robust model for disease diagnosis and lesion localization requires a large amount of samples with fine-grained labels, which is difficult to obtain due to privacy issues and expensive labeling cost. The disclosed techniques address this problem by providing a CXR synthesis framework for data augmentation in association with improving the performance of a thoracic disease detection model. The CXR framework is able to synthesize different types of lesions associated with different types of thoracic diseases at given locations of normal CXR images, which provides extra training data with bounding box annotations of lesions. To enable realistic, high-quality lesions, an adversarial training is conducted between a lesion generator and a discriminator, where the generator is encouraged to generate indistinguishable lesions from the real ones. To deal with the large variation of different lesions in size, location, and texture, the generator utilizes both convolutional layers and transformers for generating not only fine details locally, but also plausible structures globally. In addition, the generator is equipped with a style variation module to diversify the styles of synthesis via noise injection.

Furthermore, the disclosed techniques explicitly integrate the training of the lesion generator and the detector into the same framework to form a mutual-boosting loop. In this regard, in one or more embodiments, the training of the lesion generator and the detector can be performed in an alternate manner as follows: 1) when optimizing the generator, a trained detector is used to filter easy samples based on its prediction confidence to encourage the synthesis of hard samples; 2) on the other hand, the trained generator provides CXRs with synthesized lesions as extra training data of the detection model, which improves the generalization capability of the detector. As a result, the two models boost the performance of each other in a manner of alternate training.

The effectiveness of the proposed framework has been verified on both public and private data for lesion detection, covering seven diseases, such as pneumonia, nodule, and tuberculosis.

In some embodiments, the disclosed techniques can be applied to other types of lesions associated with other types of diseases and anatomical regions of the body. The disclosed techniques can also be extended to other medical imaging modalities (e.g., magnetic resonance imaging (MRI), computed tomography (CT), and others).

The term “medical image” is used to refer to image data that depicts one or more anatomical regions of a patient. Reference to a medical image or medical image data herein can include any type of medical image associated with various types of medical image acquisition/capture modalities. For example, medical images can include (but are not limited to): radiation therapy (RT) images, X-ray (XR) images, digital radiography (DX) X-ray images, X-ray angiography (XA) images, panoramic X-ray (PX) images, computerized tomography (CT) images, mammography (MG) images (including a tomosynthesis device), a magnetic resonance imaging (MRI) images, ultrasound (US) images, color flow doppler (CD) images, position emission tomography (PET) images, single-photon emissions computed tomography (SPECT) images, nuclear medicine (NM) images, and the like.

Medical images can also include synthetic versions of native medical images such as augmented, modified or enhanced versions of native medical images, augmented versions of native medical images, and the like generated using one or more image processing techniques. In this regard, the term “native” image or “real” image is used herein to refer to an image in its original capture form and/or its received form prior to processing via one or more medical image inferencing models. The term “synthetic” image is used herein to distinguish from native images or real images and refers to an image generated or derived from a native or real image using one or more synthetic image processing techniques (e.g., synthetic lesion object generation). In some embodiments, the term “image data” can include the raw measurement data (or simulated measurement data) used to generate a medical image (e.g., the raw measurement data captured via the medical image acquisition process).

The terms “algorithm” and “model” are used herein interchangeably unless context warrants particular distinction amongst the terms. The terms “artificial intelligence (AI) model” and “machine learning (ML) model” are used herein interchangeably unless context warrants particular distinction amongst the terms. Reference to an AI or ML model herein can include any type of AI or ML model, including (but not limited to): deep learning models, neural network models, deep neural network models (DNNs), convolutional neural network models (CNNs), generative adversarial neural network models (GANs) and the like. An AI or ML model can include supervised learning models, unsupervised learning models, semi-supervised learning models, combinations thereof, and models employing other types of ML learning techniques. An AI or ML model can include a single model or a group of two or more models (e.g., an enable model or the like).

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

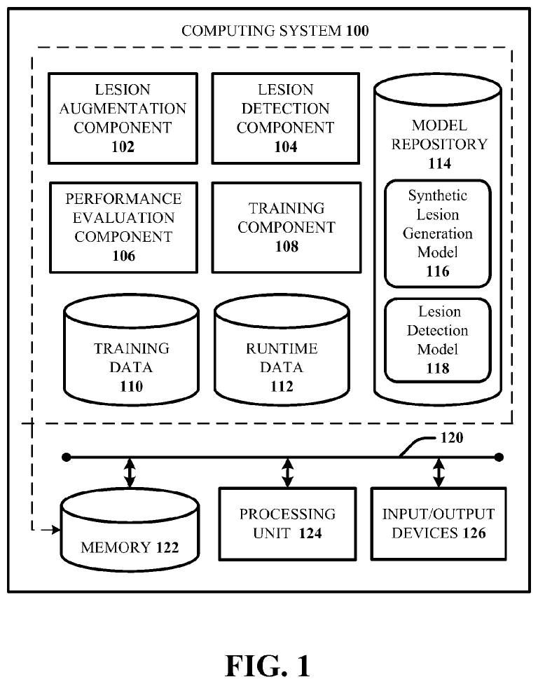

Turning now to the drawings, FIG. 1 presents an example, non-limiting computing system 100 that facilitates augmenting CXR images with synthetic lesions and employing the augmented images in association with optimizing a thoracic disease detection model, in accordance with one or more embodiments of the disclosed subject matter.

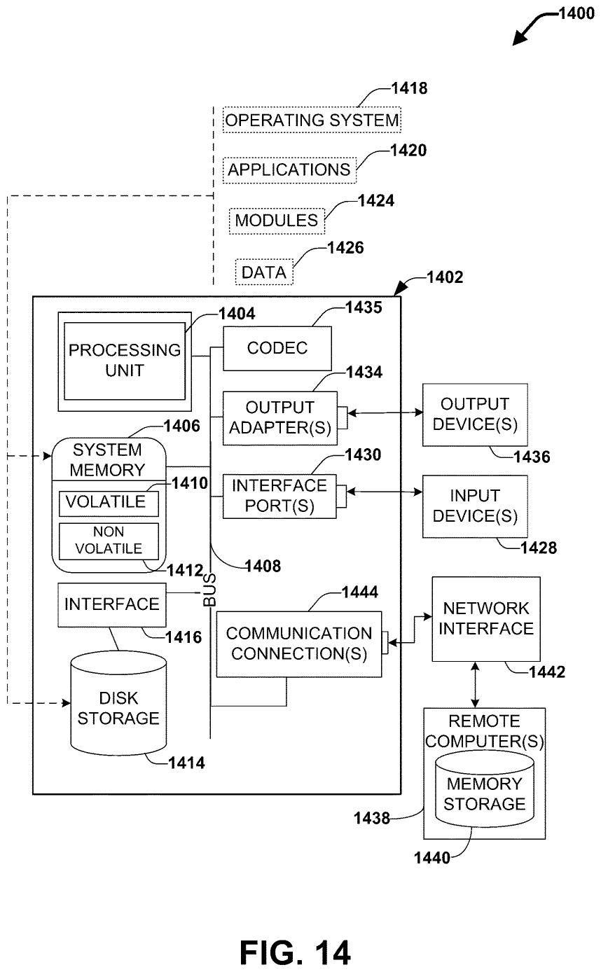

Embodiments of systems and devices described herein can include one or more machine-executable (i.e., computer-executable) components or instructions embodied within one or more machines (e.g., embodied in one or more computer-readable storage media associated with one or more machines). Such components, when executed by the one or more machines (e.g., processors, computers, computing devices, virtual machines, etc.) can cause the one or more machines to perform the operations described. These computer/machine executable components or instructions (and others described herein) can be stored in memory associated with the one or more machines. The memory can further be operatively coupled to at least one processor, such that the components can be executed by the at least one processor to perform the operations described. In some embodiments, the memory can include a non-transitory machine-readable medium, comprising the executable components or instructions that, when executed by a processor, facilitate performance of operations described for the respective executable components. Examples of said and memory and processor as well as other suitable computer or computing-based elements, can be found with reference to FIG. 14 (e.g., processing unit 1404 and system memory 1406 respectively), and can be used in connection with implementing one or more of the systems or components shown and described in connection with FIG. 1, or other figures disclosed herein.

In this regard, in one or more embodiments, computing system 100 can include (or be operatively coupled to) at least one memory 122 that stores computer-executable components and at least one processor (e.g., processing unit 124) that executes the computer-executable components stored in the at least one memory 122. The computer-executable components can include (but are not limited to), lesion augmentation component 102, lesion detection component 104, performance evaluation component 106 and training component 108. Memory 122 can further include (e.g., store) a model repository 114, training data 110 and runtime data 112. Additionally, or alternatively, the model repository 114, the training data 110, and/or the runtime data 112 may be associated with one or more additional information storage structures (e.g., transitory memory devices, non-transitory memory devices, or the like), that may be coupled to the computing system 100 either directly or via one or more wired or wireless communication networks.

The model repository 110 can include one or more models (e.g., ML/AI models and/or other types of models or algorithms) employed by the computing system 100, including both untrained versions of the models and trained versions of the models. These models can include (but are not limited to), a synthetic lesion generation model 116 and a lesion detection model 118. The training data 110 can include the training data used by the training component 108 to train and/or re-train or update the synthetic lesion generation model 116 and the lesion detection model 118. The runtime data 114 can include the runtime data (or test data) processed by the trained versions of the synthetic lesion generation model 116 and the lesion detection model 118 after at least some training has been completed.

The computing system 100 can further include one or more input/output devices 126 to facilitate receiving user input in association with training and/or updating the synthetic lesion generation model 116 and/or the one or more lesion detection model 118, and/or applying the trained versions of the respective models to the corresponding runtime data 114. In this regard, any information received by, generated by and/or accessible to the computing system 100 (e.g., training data 112, runtime data 114, synthetic lesion objects, synthetic lesion images comprising the synthetic lesion objects, annotation data, lesion detection model output results, user feedback, etc.) can be presented or rendered to a user via a suitable output device, such as a display, a speaker or the like, depending on the data format. Suitable examples of the input/output devices 122 are described with reference to FIG. 14 (e.g., input devices 1428 and output device 1436). The computing system 100 can further include a system bus 116 that couples the memory 118, the processing unit 120 and the input/output devices 122 to one another.

In one or more embodiments, the lesion augmentation component 102 can generate synthetic lesion images comprising synthetic lesion image data objects integrated on or within medical images using the synthetic lesion generation model 116, wherein the synthetic lesion generation models 116 comprises a lesion generator model (e.g., lesion generator 306) trained to generate the synthetic lesion image data objects and tailor the synthetic lesion image data objects to account for different types of lesions, different anatomical locations of the lesions, different sizes of the lesions, and different textures of the lesions. In various embodiments, the medical images and the synthetic lesion images include or correspond to medical images depicting the thoracic region (i.e., the chests) of human subjects and the different types of lesions correspond to different types of thoracic diseases or conditions. In some embodiments, the modality of the medical images and the synthetic lesion images (as well as the synthetic lesion image data objects) is XR (e.g., the medical images correspond to CXRs). In other embodiments, the modality of the medical images, the synthetic lesion images (as well as the synthetic lesion image data objects) can be CT, MRI, or another medical imaging modality.

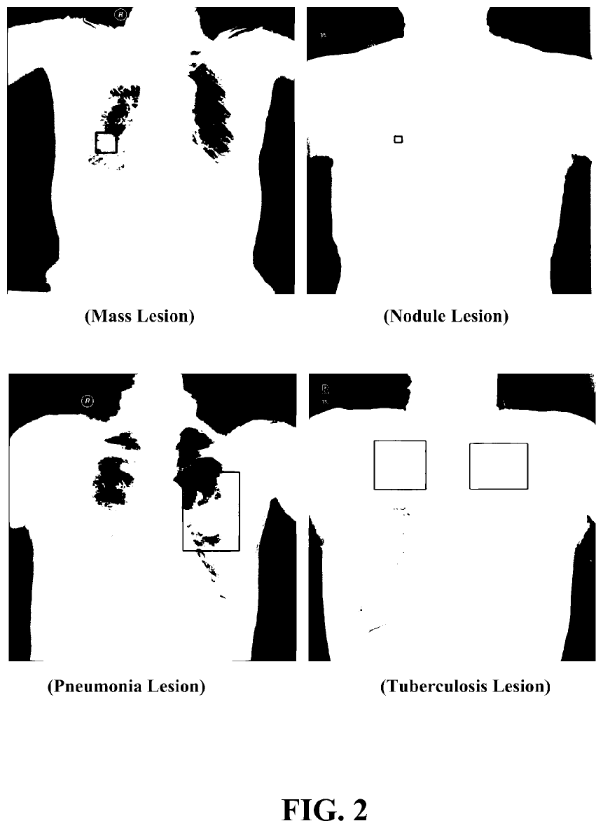

With reference to FIG. 2 in view of FIG. 1, FIG. 2 presents example CXRs with different types of lesions (e.g., a mass type lesion, a nodule type lesion, a pneumonia type lesion, and a tuberculosis type lesion) associated with different types of thoracic diseases or conditions. The example CXRs are real or native medical images depicting real lesions (as opposed to synthetic lesions generated by the synthetic lesion generation model 116). The respective lesions are indicated via the rectangular bounding boxes overlaid on the respective CXRs. In this regard, the term “lesion” is used herein to refer to a defined anatomical area of abnormal or altered tissue due to disease or injury. Although four different types of lesions are shown in FIG. 1, the disclosed techniques can be applied to various other types of lesions associated with various additional or alternative thoracic diseases and conditions (e.g., pneumothorax, pleural effusion, fracture, and others).

As shown in FIG. 1, different types of lesions associated with different types of thoracic diseases/conditions vary in location (i.e., anatomical location relative to one or more anatomical structures of the thoracic region of the body), size (e.g., wherein the size of the respective lesions corresponds to the sizes of the respective bounding boxes as illustrated in FIG. 2) texture and appearance. In accordance with one or more embodiments, the synthetic lesion generation model 116 can be configured to generate synthetic versions of the lesions shown in FIG. 2 and other types of thoracic disease/conditions lesions, in a manner such that the model accounts for the variability in the type (i.e., lesion/disease type), location, size and texture of the different types of lesions associated with different types of thoracic diseases/conditions. This task requires the synthetic lesion generation model 116 to be powerful in capturing both local details and global plausibility, as it is challenging to generate high-quality lesions of different diseases due to the large variations.

To facilitate this end, in one or more embodiments, rather than generating an entire image, the synthetic lesion generation model 116 can be configured to generate synthetic versions of only the lesions themselves and/or the portions of the images comprising the lesions. The synthetic lesions are further integrated on or within normal medical images without lesions (e.g., normal CXRs) to generate synthetic lesion images comprising the synthetic images. For example, the synthetic lesions can respectively correspond to image data objects that can be overlaid onto normal images at specified locations on or within the normal images to generate synthetic lesion images comprising lesions at the specified locations. In this way, computing system 100 can leverage a large amount of normal medical images (e.g., normal CXRs) while focusing on lesion synthesis.

In some implementations of these embodiments, the synthetic lesion generation model 116 can be configured to combine the synthetic lesion image data objects with the normal images to generate the synthetic lesion images. In other implementations, the synthetic lesion generation model 116 can be configured to output synthetic lesion image data objects and the lesion augmentation component 102 can overlay the synthetic lesion image data objects onto the normal images at the specified locations to generate the synthetic lesion images. In either of these cases, the synthetic lesion image data objects generated by the synthetic lesion generation model 116 are tailored to account for the specific type of lesion of a plurality of different lesion types and a specific anatomical location of the lesion. In this regard, once the synthetic lesion generation model 116 has been trained (e.g., via the training component 108 as discussed in greater detail below), the input to the model can include a specified lesion type and a specified anatomical location for integrating the lesion on or within a normal medical image.

For example, as applied to CXRs, in various implementations, the input to the synthetic lesion generation model 116 can include selection of a specific type of lesion of a plurality of predefined types respectively associated with different types of thoracic diseases/conditions (e.g., pleural effusion, mass, nodule, pneumonia, pneumothorax, tuberculosis, and fracture). The input can also include a specified location on or within a normal CRX for integrating the synthetic lesion. The synthetic lesion image data object generated by the model based on such input will be tailored to reflect the selected type and location. For example, the visual appearance properties (e.g., texture, content, geometry/shape, coloration, resolution, pixelation, brightness, etc.) of the synthetic lesion image data objects can vary for different types of lesions. In addition, the visual appearance properties of a same lesion type can vary for different anatomical locations selected for integration of the synthetic lesion at input. For example, the visual appearance properties (e.g., texture, content, geometry/shape, coloration, resolution, pixelation, brightness, etc.) of a synthetic lesion image data object of a same lesion type can vary for different anatomical locations selected for integration of the synthetic lesion at input. In this regard, in association with training the synthetic image generation model 116, the model can learn not only variances in visual properties of different types of lesions, but variances in visual properties of the same type of lesion at different anatomical locations.

In addition, the synthetic lesion image data objects generated by the synthetic lesion generation model 116 can be tailored to account for different lesion sizes. In this regard, the input to the synthetic lesion generation model 116 can also include a specified lesion size (e.g., which may be user specified via a mask and/or bounding box annotation applied to the medical image to which the synthetic lesion will be integrated), and the synthetic lesion generation model 116 can generate a synthetic lesion object having a size corresponding to the specified size. In addition, the visual appearance properties of the synthetic lesion can vary to account for variances in the same type of lesion at the same anatomical location of different sizes. In this regard, in association with training the synthetic image generation model 116, the model can learn variances in visual properties of the same type of lesion at the same anatomical location but at different sizes.

In this regard, it should be appreciated that the synthetic image generation model 116 can include or correspond to one or more generative machine learning models that can be trained to generate synthetic versions of lesions of different types corresponding to different thoracic diseases accounting for variations in the different types of lesions based on type, location, and size. The lesion augmentation component 102 and/or the synthetic lesion generation model 116 can further combine the synthetic lesions (e.g., synthetic lesion image data objects) with normal images (e.g., normal CRXs without lesions, included in the runtime data 112) at their respective input specified locations to generate a plurality of synthetic lesion images. The plurality of synthetic lesion images can provide a wide distribution of different types of lesion images comprising synthetic lesion image data objects with variability in lesion type, location and size. Additional details regarding the lesion augmentation component 102 and synthetic lesion generation model 116 are described infra with reference to FIGS. 2-7.

In one or more embodiments, the synthetic lesion images generated by the lesion augmentation component 102 using the synthetic image generation model 116 can be used to train (e.g., via the training component 108) a lesion detection model 118 to detect lesions depicted in the synthetic lesion images. In this regard, the synthetic lesion images can be used for image data augmentation to increase the diversity and amount of training lesion images available for training the lesion detection model 118, which improves the generalization capability of model. For example, in some embodiments, the lesion detection model 118 can include or correspond to one or more machine learning models trained to detect and classify different types of lesions corresponding to the different types of lesions represented in the synthetic lesion images. For instance, as applied to CXRs, the lesion detection model 118 can include or correspond to one or more deep learning models (or other types of machine learning models) configured to detect and classify different types of lesions corresponding to different types of thoracic diseases/conditions (e.g., pleural effusion, mass, nodule, pneumonia, pneumothorax, tuberculosis, and fracture) in input CRX images. In some embodiments, the lesion detection model 118 can also be trained to determine the size of a detected lesion and generate a confidence score that represents a measure of confidence the lesion detection model has in its inference output accuracy.

In various embodiments, the lesion detection model 118 can be trained (e.g., via training component 108) using a training dataset (e.g., included in training data 110) that includes lesion images paired with ground truth annotation information that indicates the type of lesion and the size of the lesion. The lesion images can include real lesion images (e.g., real CXRs with real lesions) and/or the synthetic lesion images generated via the synthetic lesion generation model 116. In this regard, in one or more embodiments, the lesion augmentation component 102 can employ the synthetic lesion generation model 116 to generate synthetic lesion images comprising synthetic lesions over normal CXRs (e.g., included in the runtime data 112) and add the synthetic lesion images to a training dataset (e.g., included in training data 110) for utilization in training the lesion detection model 118 (e.g., by the training component 108). The training dataset for the lesion detection model 118 can also include normal images (e.g., without lesions to train the model to correctly infer when an input image does not depict a lesion).

The training process can follow conventional supervised and/or semi-supervised machine learning processes wherein the lesion detection model 118 is trained to predict whether an input image depicts a lesion and if so, the type and size of the lesion, using one or more loss functions that assess loss (e.g., detection loss) based on comparison of the inference output with the ground truth annotation data associated with (at least some) of the input images. In this regard, because the synthetic lesion generation model 116 is trained to generate augmented lesion images with input knowledge identifying the type, location and (in some implementations) size of the lesion to be generated and applied to a normal CRX image, the augmented lesion images not only increase the amount and diversity of the training lesion images used to train the lesion detection model 118, but further include the requisite ground truth annotation data already applied/associated therewith.

In some embodiments, the lesion detection component 104 can apply the trained version of the lesion detection model 118 to new medical images included in the runtime data 112 to generate the corresponding inference output results. For example, the lesion detection component 104 can execute the lesion detection model 118 in a test phase of the training process and/or execute the lesion detection model 118 in real clinical workflows on real patient images (e.g., real CXRs). In one or more embodiments as applied to CXRs and detection of different types of thoracic disease lesions, the inference output results can include information identifying whether one or more lesions are detected in the input CXR, the detected lesion type (or thoracic disease type) of amongst a plurality of defined different types (if a lesion was detected), the detected lesion size (e.g., if a lesion was detected) and a confidence score that indicates a measure of confidence associated with the inference output results for the given input image.

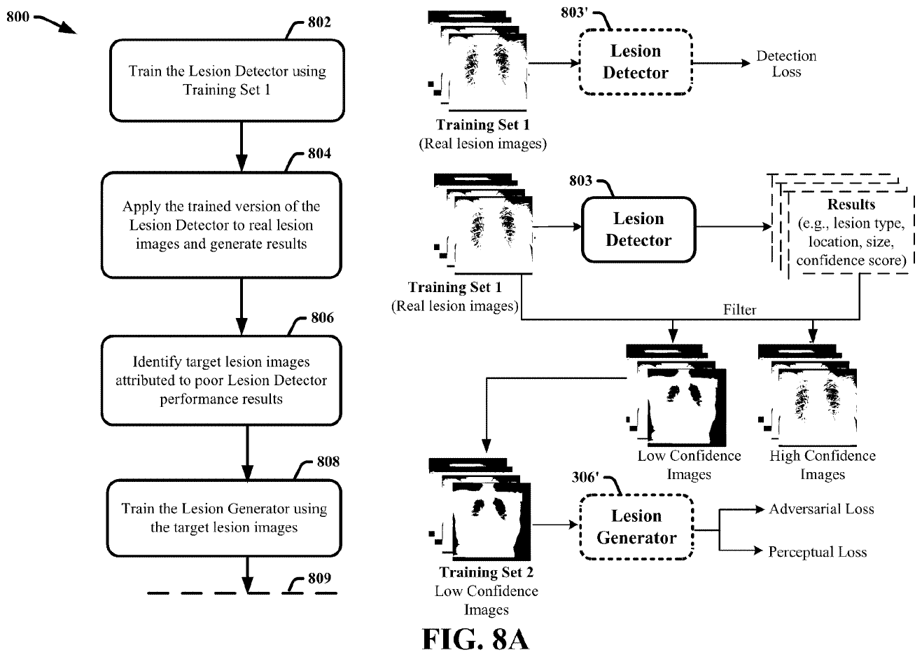

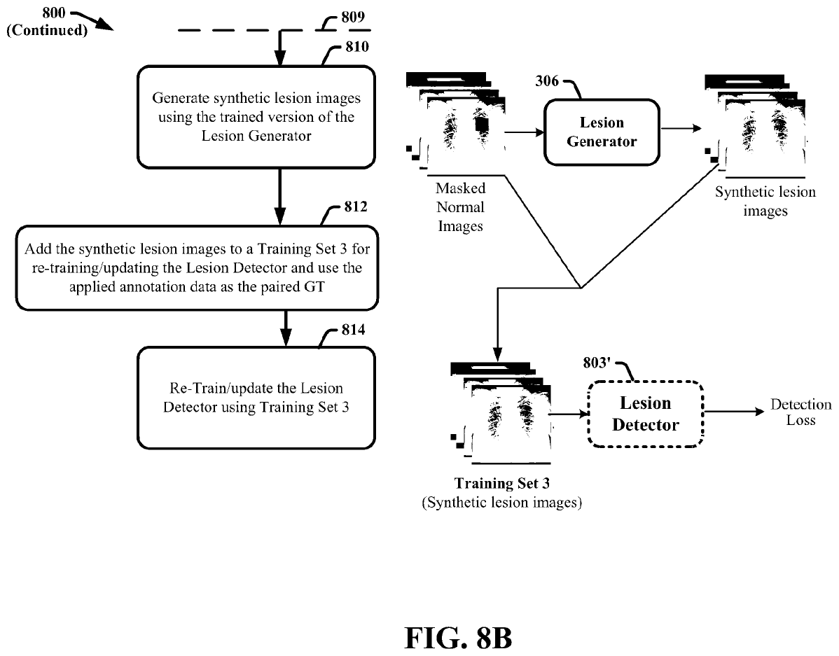

In one or more additional embodiments, the training of the synthetic lesion generation model 116 and the lesion detection model 118 can be integrated into the same framework for mutual boosting. Specifically, when optimizing (e.g., retraining/updating) the synthetic lesion generation model 116, a trained version of the lesion detection model 118 can be used to filter “easy” samples based on its prediction confidence to encourage the synthesis of “hard” samples. In this regard, reference to an easy sample refers to an input lesion image that the trained version of the lesion detection model 118 demonstrates good performance (e.g., measured as a function of a high confidence level or another performance evaluation criterion indicative of an acceptable level of model performance accuracy and/or confidence). Likewise, reference to a hard sample refers to an input lesion image that the trained version of the lesion detection model 118 demonstrates poor performance (e.g., measured as a function of a low confidence level or another performance evaluation criterion indicative of an unacceptable level of model performance accuracy and/or confidence). With these embodiments, the performance evaluation component 108 can facilitate evaluating the performance of a trained version of the lesion detection model as applied to one or more lesion images (e.g., real lesion CXRs included in the runtime data 112) by the lesion detection component 104 in association with identifying a subset (e.g., including one or more) of the runtime lesion images for which the performance of the lesion detection model 118 is considered inaccurate or insufficient (e.g., based on a low confidence score or another performance evaluation criterion). For example, the performance evaluation component 106 can identify any input lesion images processed by the trained version of the lesion detection model 118 that received a confidence score below a threshold confidence score. The performance evaluation component 106 can further add the subset of lesion images to a new training data set (e.g., included in the training data 110) and the training component 108 can further retrain or update the synthetic lesion generation model 116 using the identified subset of lesion images added to the new training dataset. Additional details regarding the alternate training of the synthetic lesion generation model 116 and the lesion detection model 118 are provided infra with reference to FIGS. 8A-10.

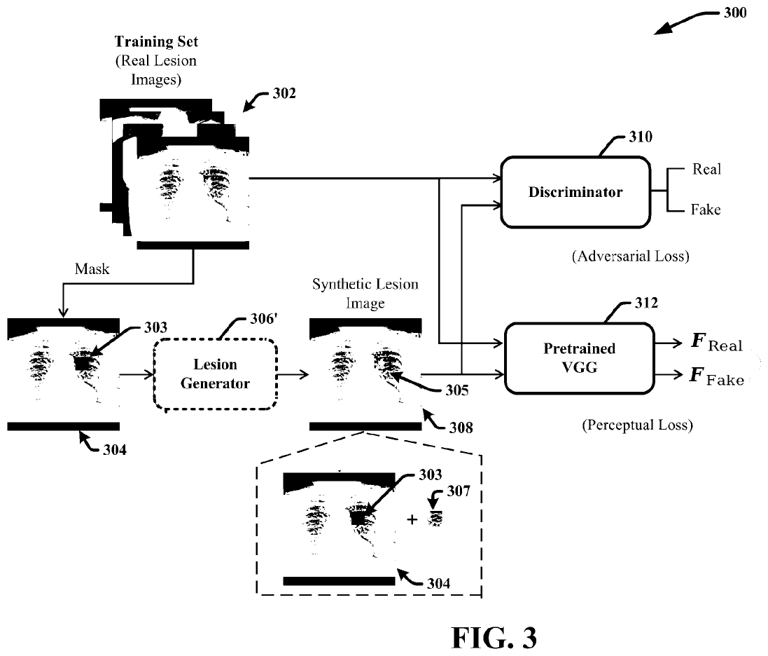

FIG. 3 presents an example process 300 for training the synthetic lesion generation model 116 in accordance with one or more embodiments of the disclosed subject matter. With reference to FIGS. 1-3, in one or more embodiments, the synthetic lesion generation model 116 can employ a GAN-based framework which employs an adversarial training process between a lesion generator 306′ and a discriminator 310. During training, the lesion generator 306′ learns to generate synthetic lesion images (e.g., synthetic CXRs) that mimic the distribution of real lesion images of the training set 302 (e.g., real CXRs with real lesions of various types, locations and sizes), while the discriminator 310 learns to distinguish between the real lesion images and synthetic (or “fake”) lesion images. Once training has been completed (e.g., after convergence has been reached and/or the loss has reached an acceptable level), the trained version of the lesion generator 306′ can be applied to normal CXRs (e.g., real CXRs without lesions) by the lesion augmentation component 102 to generate synthetic lesions over the normal CXRs at specified locations and sizes to generate the synthetic lesion images comprising the synthetic lesions. In this regard, in various embodiments, the synthetic lesion generation model 116 can include or correspond to the lesion generator 306′ (or vice versa).

In accordance with the remaining description and Figures, unless otherwise specified, an apostrophe and a dashed line is used to indicate a version of a model under training and a sold line for the same model with the same reference number minus the apostrophe (e.g., lesion generator 306 as opposed to lesion generator 306′) is used to indicate a trained version of the model.

As noted above, in one or more embodiments, the training component 108 does not train the synthetic lesion generation model 116 (or more specifically the lesion generator 306′/306) to directly generate an entire synthetic CXR image but rather generate only the region of the image comprising the lesion (e.g., also referred to as the lesion area). In particular, the lesion generator 306′ focuses on synthesizing only the lesion areas by taking a masked CXR 304 as input, wherein the masked CXR 304 corresponds to a real CXR from the training set with a real lesion and a mask 303 formed over the real lesion with a mask size corresponding to the size of the real lesion. In this regard, in association with generating the synthetic lesion image 308, the lesion generator 306′ is trained to “fill in” the masked area 305 of the masked CXR 304 with a synthetic lesion image data object 307. For example, the synthetic lesion image 308 corresponds to the masked CXR 304 with the mask 303 removed and replaced with the synthetic lesion image data 307. (The masked area 305 is indicated on the synthetic lesion image 308 for exemplary purposes. In practice, the masked area 305 is not marked on the synthetic lesion images generated by the lesion generator 306.) In this regard, the lesion generator 306′ can be trained to generate a synthetic lesion image data object 307 for a masked area 305 of an input image and integrate the synthetic lesion image data 307 over the masked area 305 to generate a synthetic lesion image 308. Meanwhile, the discriminator 310 is trained to distinguish between the synthetic lesion image 308 and the corresponding real version of the input image (i.e., masked CXR 304 with the mask 303 removed). In this manner, the mask 303 defines the location and the size of the synthetic lesion image data object 3057 for generation by the lesion generator 306′. The lesion generator also takes, as input, the specific type of lesion covered by the mask 303 that the lesion generator 306′ is expected to generate. The training set 302 can include real lesion images with a variety of different types of lesions (e.g., corresponding to different types of thoracic diseases) at different locations and of different sizes.

As illustrated in FIG. 3, in some embodiments, the lesion generator 306′ can be trained based on both adversarial loss (e.g., as function of the discriminator 310) and perceptual loss (e.g., using a pretrained VGG 312). The discriminator 310, the pretrained VGG 312 and the corresponding parameter settings can be stored and accessed by the training component 108 in the model repository 114. The adversarial loss encourages the lesion generator 306′ to generate realistic lesions, while the perceptual loss eases the adversarial training by fitting the high-level perceptual information of the image. In one or more embodiments, the loss functions of the lesion generator 306′ (L_{G}) and the discriminator 310 (L_{D}) can respectively be formulated using Equations 1 and 2 below where x and \hat{x} represent the real and generated CXR, respectively.

$${L}_{G} = - {E}_{\hat{x}} [ log ( D ( \hat{x} ) ) ] \qquad ( Equation 1 )$$

$${L}_{G} = - {E}_{\hat{x}} [ log ( D ( \hat{x} ) ) ] - {E}_{\hat{x}} [ log ( 1 - D ( \hat{x} ) ) ] \qquad ( Equation 2 )$$

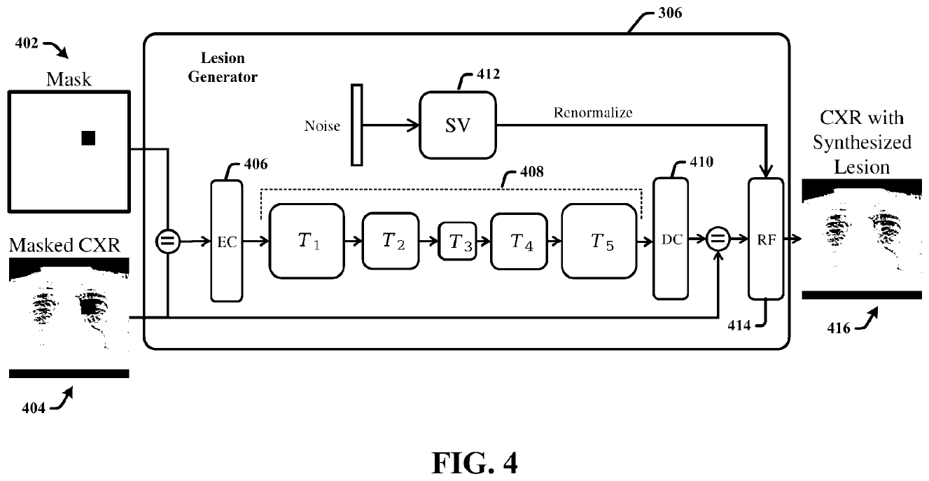

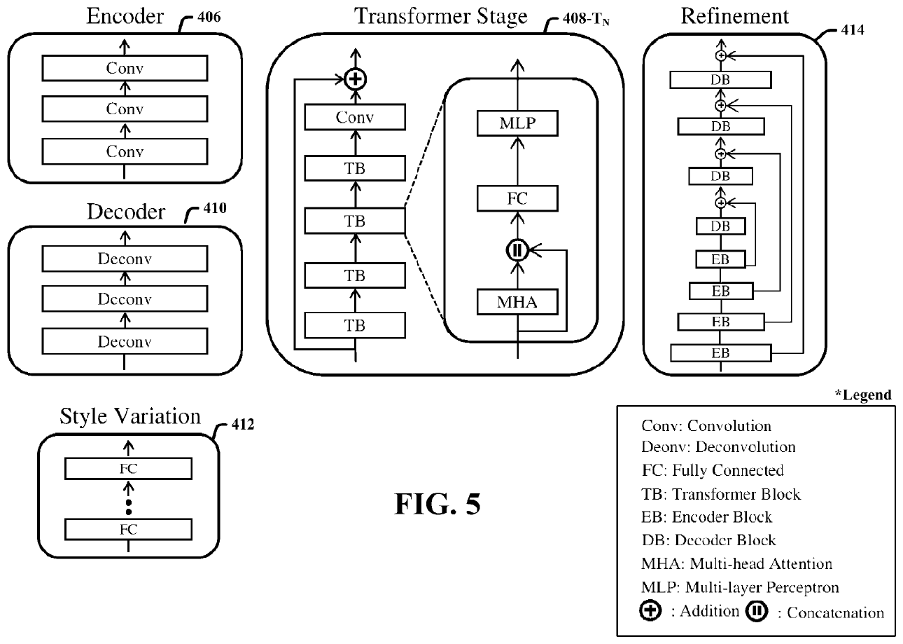

In one or more embodiments, the discriminator 310 can include seven convolutional layers and two fully connected (FC) layers for binary classification, where each convolutional layer has kernel size 3×3 and stride 2. Additional details regarding the lesion generator 306′ are described infra with reference to FIGS. 4 and 5.

In some embodiments, a regularization R₁ can be used to improve the quality and stability of the generated images by regularizing the discriminator 310. With these embodiments, the regularization R₁ can be applied to the discriminator 310 and formulated in accordance with Equation 3, where ∇D(x) is the gradient of the discriminator.

$$\text{?} = {E}_{x} ∇ D \text{?} \qquad ( Equation 3 )$$

$$\text{?} \text{indicates text missing or illegible when filed}$$

The perceptual loss can be incorporated into the training of the lesion generator 306′ to take into account high-level perceptual information about an image, rather than just pixel-level differences. By incorporating perceptual loss into the training process of a GAN, the training component 108 can encourage the lesion generator 306′ to produce images that not only look visually pleasing but also have higher-level semantic meaning. This can lead to more realistic and diverse generated images, as well as better preservation of details and textures in the original image. The perceptual loss (L_{P}) can be formulated in accordance with Equation 4, where φ represents the conv5_4 layer of an ImageNet pretrained VGG model 312.

$${L}_{p} = {‖ φ ( \hat{x} ) - φ ( x ) ‖}_{1} \qquad ( Equation 4 )$$

Thus, in one or more embodiments, the loss function L used to train the lesion generator 306′ can be formulated in accordance with Equation 5, where α and β are balancing coefficients and wherein the values can be selected/adapted as desired. In example implementations, we set α=10 and β=0.1.

$$L = \text{?} + \text{?} + \text{?} \qquad ( Equation 5 )$$

$$\text{?} \text{indicates text missing or illegible when filed}$$

FIG. 4 presents a more detailed illustration of the lesion generator 306 in accordance with one or more embodiments of the disclosed subject matter. As illustrated in FIG. 4, the lesion generator 306 corresponds to a trained version of the lesion generator. As noted above, the input to the lesion generator 306 can include a mask 402 applied to a normal CXR, resulting in masked CXR 404. The applied mask 402 defines the size and location of the synthetic lesion image data object to be generated and integrated over the normal CXR. The input to the to the lesion generator 306 also includes a selected lesion type of a plurality of defined lesion types corresponding to different types of thoracic diseases/conditions. The output of the lesion generator 306 includes a synthetic lesion image 416; that is a CXR with a synthesized lesion (e.g., a synthetic lesion image data object) overlaid onto the normal CRX over the region of the normal CXR covered by the mask 402.

In one or more embodiments, the lesion generator 306 can include an encoder (EC) component 406, a transformer component 408 (e.g., comprising transformer stages T₁-T₅), a decoder (DC) component 410, a refinement (RF) component 414, and a style variation (SV) component 412. FIG. 5 presents a more detailed view of these respective components of the lesion generator, (wherein 408-T_{N}, can correspond to each one of T₁₋₅), in accordance with one or more embodiments of the disclosed subject matter.

With reference to FIGS. 4 and 5, in various embodiments, to produce synthetic lesions with fine details, the lesion generator 306 can utilize convolutional layers in both encoder component 406 and the decoder component 410 to deal with local texture processing and reconstruction, respectively. For example, the encoder component 406 can employ a plurality of convolutional layers to downsample and extract local features of the masked input image (e.g., masked CXR 404) and the decoder component can employ a plurality of convolutional layers 410 to up-samples and reconstruct the image. The refinement component 414 is further used to refine high-frequency details of the synthesized lesion image data. To obtain a structurally plausible image, a transformer (e.g., transformer component 408) is introduced as the main body to model the long-range interactions between local and global features. The transformer component 408 can include five stages in different resolutions (e.g., transformer stages T₁-T₅). The style variation component 412 can further impose diversity on the synthesized lesions by injecting a noise vector in the process of refinement. In this way, the lesion generator 306 is able to generate plausible synthetic lesion CXR images with realistic and diverse lesions in different styles. A detailed description of the components of the lesion generator 306 in accordance with one or more embodiments is outlined below with reference to FIGS. 4 and 5.

### Encoder and Decoder Components:

In one or more embodiments, the input CXR image can be denoted as [∈R^{C×H×W}, and the mask 402 (a binary mask) applied to the image can be denoted as M∈{0.1}^{C×H×W}, where C, H, and W represent the length of channel, height, and width, respectively. Then, the masked CXR 404 can be obtained and defined as CXR [_{M}=I⊙M, where ⊙ denotes element-wise product. The masked region can be defined by a zero value and indicates the location where the synthesized lesion is to be generated and applied by the lesion generator 306. The encoder component 406 takes as input the concatenation of I_{M} and M, then downsamples it to the ⅛ of the original size with C′ channels via three convolutional layers. Each convolutional layer can have kernel size 3×3 and stride 2. The decoder component 410 upsamples the output of the transformer component 408 to the same resolution as the input with three deconvolutional layers. Each deconvolutional layer can have kernel size 4×4 and stride 2.

Transformer Component: As illustrated in FIG. 4, in one or more embodiments, the transformer component 408 can include five transformer stages respectively indicated as T₁, T₂, T₃, T₄, and T₅. As shown in FIG. 5, (wherein 408-T_{N}, can correspond to each one of T₁₋₅), each transformer stage can include four transformer blocks and a convolutional layer with residual connection. Within each transformer block (TB), the input first goes through a multi-head attention (MHA), then its output is concatenated with the input to further go through a fully connected (FC) layer and a multi-layer perceptron (MLP). Formally, for the l-th block in the k-th stage, can be defined in accordance with Equations 6 and 7 below where the input x_{k,i-1} is from the previous block and the output is Xₖᵢ.

$$\text{?} = FC ( [ MHA ( \text{?} ) , \text{?} ] ) \qquad ( Equation 6 )$$

$$\text{?} = MLP ( \text{?} ) \qquad ( Equation 7 )$$

$$\text{?} \text{indicates text missing or illegible when filed}$$

To produce hierarchical representations with an efficient attention mechanism, the MHA can employ a shifted window design, which is formulated in accordance with Equation 8, where Q. K. V∈R^{M}^{²}^{×d} are query, key, and value matrices; d is the length of these embeddings and M′ is the number of patches in a window.

$$MHA ( Q , K , V ) = Softmax ( \text{?} ) V \qquad ( Equation 8 )$$

$$\text{?} \text{indicates text missing or illegible when filed}$$

### Refinement Component:

As illustrated in FIG. 5, in one or more embodiments, the refinement component 414 can start with four encoder blocks for downsampling. In some embodiments, each encoder block (EB) is a residual block composed of convolutional layers with stride 2. Then the output of the last encoder block can be upsampled by four decoder blocks (DBs), each of which can also be a residual block based on deconvolutional layers, with stride 2. In one or more embodiments, there are skip connections between the encoder and decoder blocks that are of the same resolutions. Such connections allow the direct transfer of information from the encoder to the decoder and ensure that important details are preserved throughout the reconstruction process. Moreover, the multi-scale manner of the refinement module also ensures that the final reconstructed image has accurate and fine-grained details at all levels of resolution. In various embodiments, the weights of the convolutional and deconvolutional layers of the refinement component 414 are further renormalized by the style variation component as described below.

### Style Variation Component:

In one or more embodiments, the lesion generator 306 can employ the style variation component 412 to encourage the diversity of the synthetic lesion image data objects. The style variation component 412 can change the style of the synthetic lesion image data objects by renormalizing the weights of the convolutional and deconvolutional layers of the refinement component 414 with a style vector S∈R^{d} during the process of refinement, where s is controlled by a random noise in accordance with Equation 9, where z E R is a random vector from Gaussian distribution, and SV is a mapping function that consists of six FC layers.

$$S = SV ( z ) \qquad ( Equation 9 )$$

After the mapping, the renormalization can be performed using Equations 10 and 11, where i, j, and k represent the indices of input channel, output channel, and spatial position of the convolution, respectively; E is a small constant to avoid division by zero.

$$\text{?} = \text{?} \qquad ( Equation 10 )$$

$$\text{?} = \text{?} \qquad ( Equation 11 )$$

$$\text{?} \text{indicates text missing or illegible when filed}$$

By modulating the convolutional and deconvolutional weights of the refinement component 414 with the style vector s, the lesion generator 306 is able to produce lesions with diverse styles.

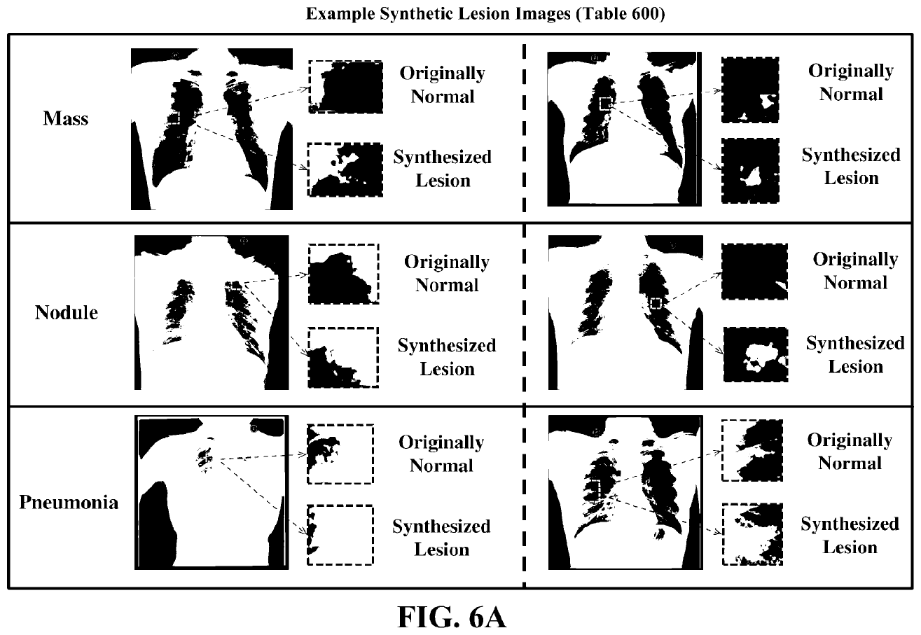

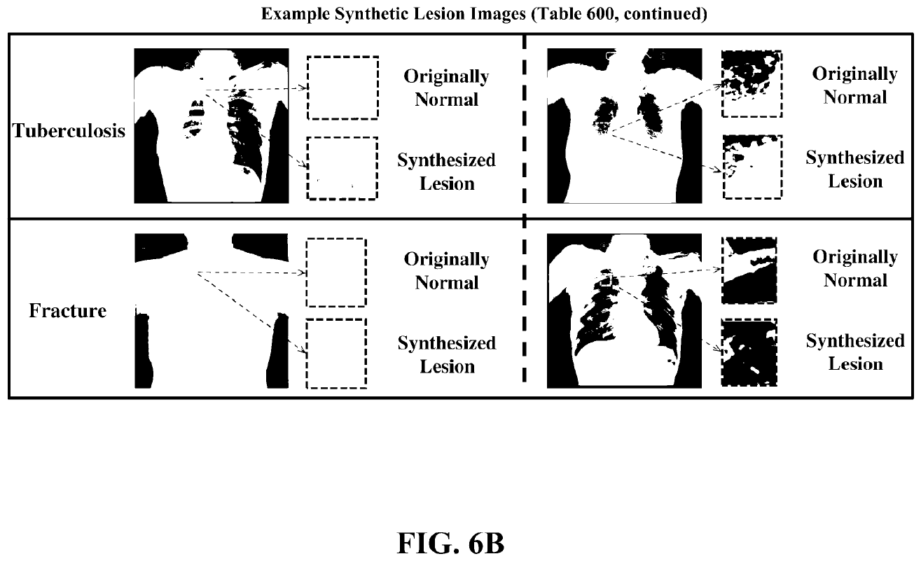

FIGS. 6A and 6B present a table (Table 600) illustrating example synthetic lesion images generated by a synthetic lesion generation model (e.g., synthetic lesion generation model 116 comprising lesion generator 306) in accordance with one or more embodiments of the disclosed subject matter. In particular, Table 600 illustrates ten different synthetic lesion CXRs (e.g., wherein the full sized CXRs correspond to synthetic lesion CXRs) generated be a trained version of the lesion generator 306 in accordance with method 300 and wherein the lesion generator 306 employs the architecture described above with reference to FIGS. 4 and 5. The ten different synthetic lesion CXRs include two different examples for each of five different types of thoracic disease/condition lesions; mass, nodule, pneumonia, tuberculosis, and fracture. Each of the ten different synthetic lesion CXRs were generated by the lesion generator 306 using various different normal CXRs (e.g., CXRs without lesions) as input with masks applied to the normal CXRs to indicate the lesion areas (e.g., defining the lesion location and size) for integration of the synthesized lesions. The input to the lesion generator 306 also indicated the desired type of lesion for integration on or within the respective lesion areas (e.g., mass, nodule, pneumonia, tuberculosis, and fracture). In Table 600 the lesion areas are marked on the synthetic lesion CXRs with bounding boxes. Enlarged views of the lesion areas as originally normal and as including the synthesized lesions (e.g., respectively corresponding to synthetic lesion image data objects) are provided to the right side of the respective synthetic lesion CXRs. As can be seen in Table 600, the disclosed techniques are able to replace a defined region of a normal CXR with synthetic lesion image data of a specific disease. The synthesized lesions not only reflect the characteristics of the different diseases, but also possess consistent semantics and textures with the surrounding areas, taking into account the lesion type, location and size. For example, of particular note, the synthetized lesions for each of the same type of disease are clearly different in appearance (e.g., texture, resolution, content, etc.), demonstrating how the lesion generator 306 tailors the appearance of the synthesized lesions to account not only for lesion type, but the selected anatomical location and size, while also ensuring the synthetic lesions possess consistent semantics and textures with the surrounding areas of the different original input CXRs used for each of the same type of disease. The powerful capability of the lesion generator 306 can be attributed to its utilization of both local details and global structures for generation of the synthesized lesions.

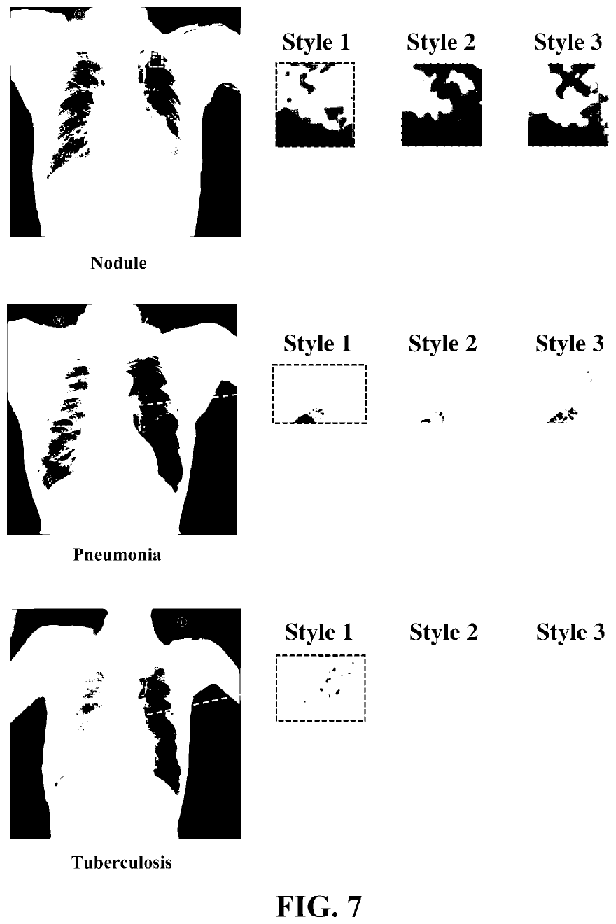

FIG. 7 presents additional example of synthetic lesion images generated in accordance with the disclosed techniques with examples of different types of styles of synthetic lesion objects capable of being generated by lesion generator 306 via the style variation component 412, in accordance with one or more embodiments of the disclosed subject matter. Similar to Table 600, FIG. 7 depicts three different synthetic lesion images generated by the lesion generator 306 (e.g., wherein the full sized images are the synthetic lesion images). The synthetic lesion images respectively correspond to CXRs with synthetic lesions of three different types (e.g., nodule, pneumonia, and tuberculosis lesions) generated on or within the regions marked by the bounding boxes overlaid on the respective CXRs. An enlarged view of the lesion areas comprising the synesthetic lesions (e.g., synthetic lesion image data objects) in three different example styles (e.g., respectively corresponding to different or random amounts of noise injection introduced via the style variation component 412) are provided to the right side of each synthetic lesion image. As can be seen by comparison of the synthetic lesions for a same lesion type in each of the three different styles, the style variation component 412 provides for generating a plurality of different synthetic lesion images using the same input CXR and input parameters (e.g., lesion type, location and size) by varying the style of the synthetic lesions. For example, the generated nodules in styles 1, 2 and 3 all have different shapes and locations, and the generated lesions of pneumonia and tuberculosis in styles 1, 2, and 3 all have different textures.

As illustrated in FIGS. 6 and 7, the proposed lesion-aware CXR synthesis framework is able to synthesize realistic and diverse lesions of various thoracic diseases, which largely mitigates the insufficient data problem in lesion detection. In this regard, as described above with reference to FIG. 1, the trained version of the synthetic lesion generation model 116 (e.g., which can include and/or correspond to the trained version of the lesion generator 306) can be used to generate a wide range of synthetic lesion images such as those illustrated in FIGS. 6 and 7, which can be used by the training component 108 to train the lesion detection model 118.

In addition, in various embodiments, the training component 108 can employ an alternate training strategy wherein the training of the lesion generator 306 is seamlessly integrated into the training of the lesion detection model 118 (also referred to as the lesion detector 803). In this regard, the inventors of the disclosed techniques argue that the training of the lesion generator should be driven by the performance of the lesion detector as the latter is the ultimate task that we want to improve. On the other hand, the trained lesion generator can improve the detection performance by providing large amount of synthesized lesion CXRs as data augmentation. Therefore, in some embodiments, the training component 108 an integrate the training of the lesion generator 306 and the lesion detector 803 to form a continuous loop such that the respective models boost the performance of each other in a manner of alternate training.

For example, in one or more embodiments, instead of training the lesion generator independently on all the available training data (e.g., real lesion CXRs), the training component 108 can use the predictions of the lesion detector as a feedback. More specifically, the training component 108 can first train the lesion detector on an original set of training data comprising real lesion CXRs. Then, the performance evaluation component 106 can filter the easy samples of the original training set if its predicted confidence score of a lesion area is larger than a threshold. The training component 108 can then train the lesion generator 306′ in accordance with process 300 using the selected hard samples. In this way, the lesion generator can be specifically trained to synthesize samples that the lesion detector does not perform well on, which can be used to update (e.g., retrain/optimize) the lesion detector. This process is further described with reference to FIGS. 8A and 8B below and process 800.

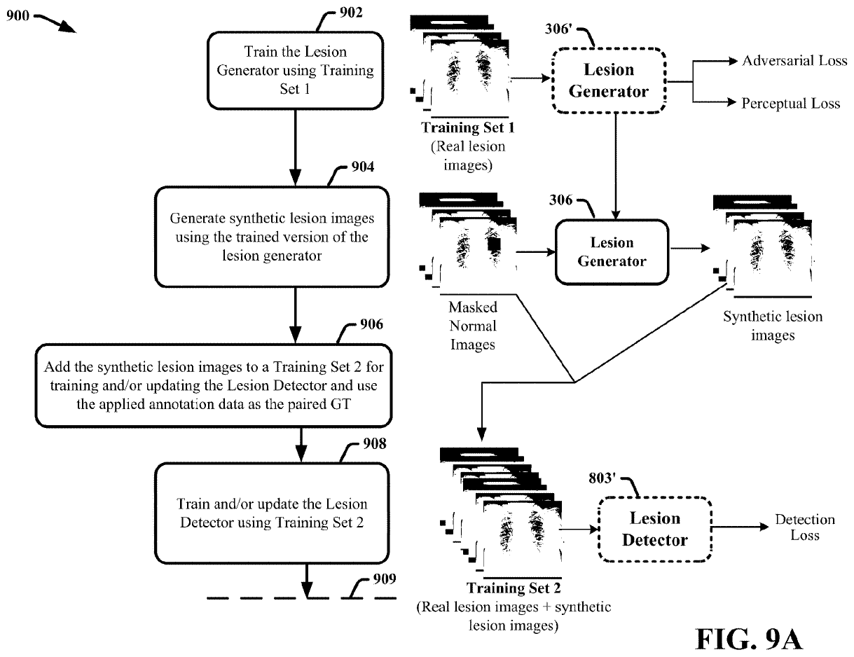

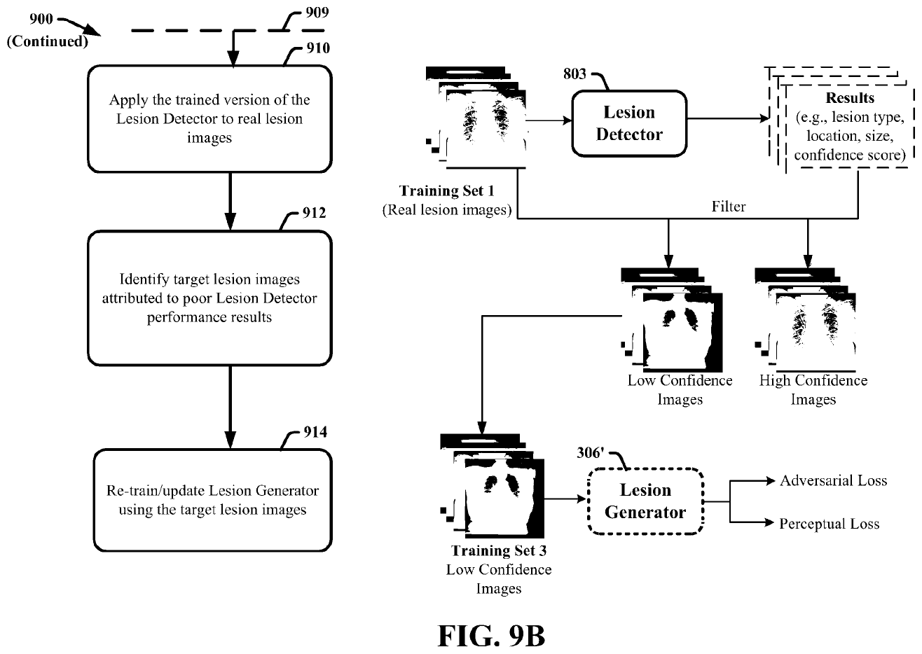

Likewise, after a trained version of the lesion generator 306 has been developed, the lesion augmentation component 102 can use it for data augmentation to boost the performance of the lesion detector. In this regard, when training the lesion detector, in addition to the original training data, the lesion augmentation component 102 can further introduce a large number of normal CXRs for lesion synthesis. More specifically, the lesion augmentation component 102 can identify the lung area of a normal CXR. The lesion augmentation component 102 can then generate a mask in rectangle within the lung area and select a specific type of lesion to be generated within the masked area. In some embodiments, the size, location, and of the mask and the lesion type can be randomly selected. In other embodiments, the size, location and aspect ratio of the mask and the lesion type can be defined based on user input. In other embodiments, to simulate the distribution of different lesions, the lesion type and the location, size, and aspect ratio of the mask are sampled from the GTs. Once the input parameters have been defined for respective normal CXRs (e.g., the lesion type, mask location, mask size, mask aspect ratio, and optionally the style variation), the lesion augmentation component 102 can apply the lesion generator 306 to synthesize a lesion on or within the masked area of the normal CXRs to generate the synthetic lesion images. The synthetic lesion images can then be used to by the training component 108 to train the lesion detector 803, using the masked area and the applied input parameters (e.g., defining the lesion type) as the ground truth annotation. In this regard, the applied mask can be used as an annotation in bounding box to define the ground truth size and location of the lesion. In this way, the proposed lesion generator helps improve the generalization capability of the detection model by providing extra augmented and annotated data. This process is further described with reference to FIGS. 9A and 9B below and process 900.

In this regard, FIGS. 8A and 8B present a flow diagram of an example process 800 for training the lesion detector 803 (e.g., wherein the lesion detection model 118 can include or correspond to lesion detector 803, and vice versa), and the lesion generator 306 in accordance with one or more embodiments of the disclosed subject matter.

With reference to FIG. 8A, in accordance with process 800, at 802, the training component 108 can train the lesion detector 803′ using a training set 1 which comprises real lesion images (e.g., real CXRs with real lesions of various types, locations and sizes). At 804, the lesion detection component 104 can apply the trained version of the lesion detector 803 to real lesion images to generate corresponding results (e.g., detection of lesion type, location, size and a confidence score indicating a measure of confidence the lesion detector 803 has in the lesion type, location and/or size being correct for a given input image). In one or more embodiments, the real lesion images used at 804 can include some or all of the real lesion images from training set 1. Additionally, or alternatively, the real lesion images used at 804 can include a new group of real CXRs with real lesions that were excluded from training set 1 (e.g., a test set or the like). At 806, the performance evaluation component 106 can identify one or more target lesion images attributed to poor performance results of the lesion detector 803 on the test set. For example, in some embodiments, the performance evaluation component 106 can filter the test set images based on their associated confidence scores and select the low confidence images (e.g., having a confidence score below a defined threshold). In other embodiments, other criteria indicative of poor model performance may be utilized to identify a subset of the test images for training the lesion generator 306′ (e.g., images belonging to a defined patient subgroup, images associated with errors attributed to artifacts, or another criterion). At 808, the training component 108 can add the target lesion images (e.g., the low confidence images) to a training set (e.g., training set 2) and employ the target lesion images to train the lesion generator 306 in accordance with process 300 (e.g., using manually applied masks over the lesions for training purposes).

Continuing process 800 from dashed line 809 in FIG. 8B, at 810, the lesion augmentation component 102 can employ the trained version of the lesion generate 306 to generate synthetic lesion images. In this regard, as described above, the lesion augmentation component 102 can obtain a set of normal CXRs (e.g., without lesion), apply masks to the images, and define the type of lesions to generate on or within the masked regions, resulting in “masked” normal images. In accordance with method 800, in some embodiments, to generate additional training images corresponding to the low confidence images (i.e., training set 2 of method 800), at 810, the lesion augmentation component 102 can control the input parameters defining the respective lesion types, locations and sizes for generation on or within the respective normal images based on the distribution of respective lesion types, locations, and sizes of the low confidence images. Additionally, or alternatively, the lesion augmentation component 102 can randomly define the input parameters while ensuring a variety of different lesion types, location and sizes are applied. Still in other implementations, the one or more of the input parameters (e.g., the lesion type, mask size, location and aspect ratio for the respective the input normal images) may by user defined (e.g., based on user input).

At 812, the training component 108 can add the synthetic lesion images to a new training set (e.g., training set 3) for re-training/updating the lesion detector and use the applied annotation data (e.g., the masks and the selected/defined lesion types) as the paired ground truth (GT). At 814, the training component 108 can then re-train/update the lesion detector 803′ using the synthetic lesion images, resulting in optimized or updated version of the lesion detector 803.

FIGS. 9A and 9B present a flow diagram of another example process 900 for training the lesion detector 803 and the lesion generator 306 in accordance with one or more embodiments of the disclosed subject matter. Repetitive description of like elements employed in respective embodiments is omitted for sake of brevity.

With reference to FIG. 9A, in accordance with metho 900, at 902, the training component 108 can train the lesion generator 306′ using training set 1 comprising a set of real lesion images (e.g., in accordance with process 300). At 904, the lesion augmentation component can generate synthetic lesion images using the trained version of the lesion generator 306 and a set of normal CXRs in association with receiving input applying masks to the normal CXRs and information defining the lesion types to be generated, as described above with reference to 810). At 906, the training component 108 can add the synthetic lesion images to new training set (e.g., training set 2) for training and/or updating the lesion detector and use the applied annotation data as the paired GT. As illustrated in FIG. 9A, in various embodiments, the synthetic lesion images can be used to augment an original set of real lesion images available for training the detector (e.g., the training set 2 can include real lesion images and the synthetic lesion images). At 908, the training component 108 can then train and/or update the lesion detector using training set 2.

Continuing with method 900 in FIG. 9B from dashed line 909, at 910, the lesion detection component 104 can apply the trained version of the lesion detector to real lesion images to generate corresponding results (e.g., detection of lesion type, location, size and a confidence score indicating a measure of confidence the lesion detector 803 has in the lesion type, location and/or size being correct for a given input image). In one or more embodiments, the real lesion images used at 910 can include some or all of the real lesion images from training set 1. Additionally, or alternatively, the real lesion images used at 910 can include a new group of real CXRs with real lesions that were excluded from training set 1 (e.g., a test set or the like). At 912, the performance evaluation component can identify target lesion images attributed to poor lesion detector performance results (e.g., low confidence images as described with reference to 806). At 914, the training component 108 can further re-train or update the lesion generator 306′ using the target lesion images (e.g., the low confidence images can be applied to a new training det and used to update the lesion generator 306′).

It should be appreciated that process 800 and/or process 900 can respectively be performed in a continuous manner to continuously update the lesion detector and the lesion generator based on the performance of the lesion detector.

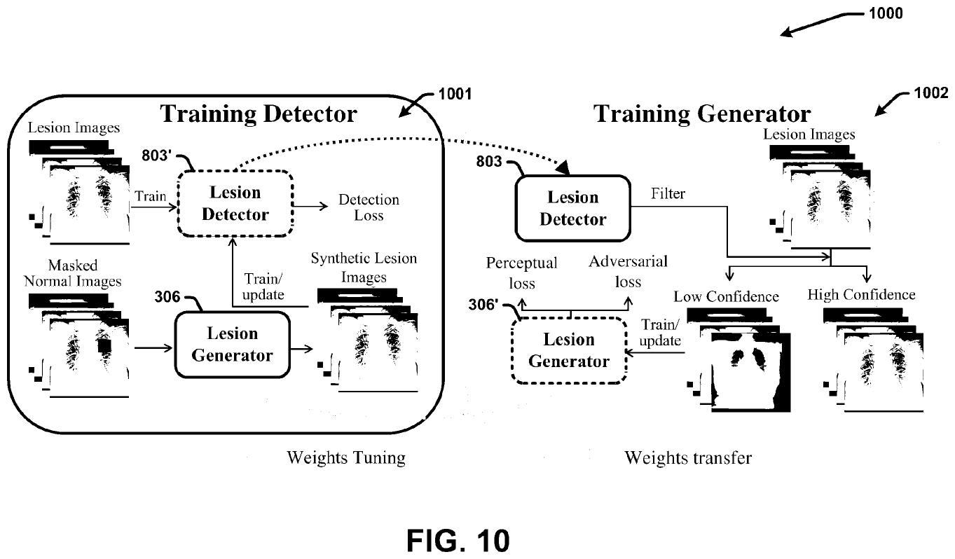

In this regard, FIG. 10 presents a high-level illustration of an alternate training framework 1000 for training the lesion generator and the lesion detector for mutual boosting in accordance with one or more embodiments of the disclosed subject matter. The alternate training framework comprises a process 1001 for training and/or updating the lesion detector 803′ using synthetic lesion images generated by a trained version of the lesion generator 306, and a process 1002 for training and/or updating the lesion generator 306′ using low confidence samples identified based on the performance of a trained version of the lesion detector 803. As illustrated in FIG. 10, process 1001 and process 102 can be connected in a continuous loop wherein the respective processes may be performed in an alternating manner to simultaneously boost performance of the respective models. In some embodiments, process 1000 may be initialized using process 1001 and thereafter proceed to process 1002 and continue in the direction indicated by the weights transfer arrows (e.g., clockwise). In other embodiments, process 1000 may be initialized using process 1002 and thereafter proceed to process 1001 and continue in the direction indicated by the weights transfer arrows (e.g., clockwise).

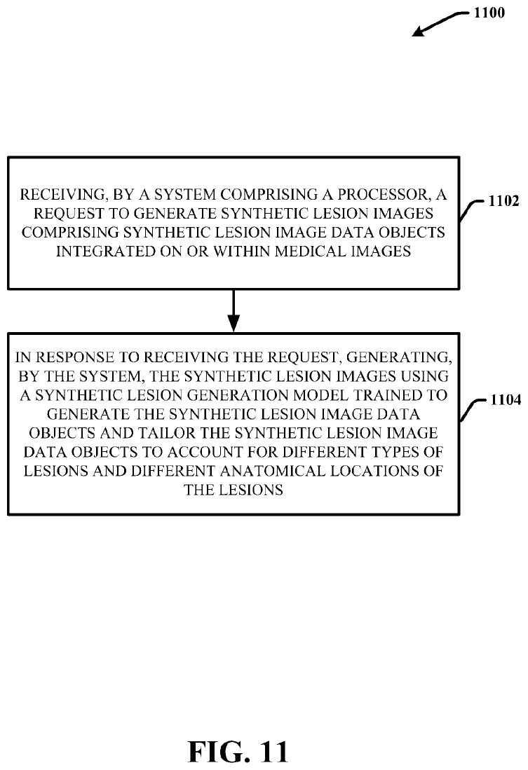

FIG. 11 illustrates a block diagram of an example, non-limiting computer implemented method 1000 for augmenting CXR images with synthetic lesions in accordance with one or more embodiments of the disclosed subject matter. Process 1100 comprises, at 1102, receiving, by a system comprising a processor (e.g., computing system 100), a request to generate synthetic lesion images comprising synthetic lesion image data objects integrated on or within medical images (e.g., via the lesion augmentation component 102). For example, the request may correspond to a request received from a user, the training component 108 (e.g., in association with directing the lesion generator 306 to generate the synthetic lesion images at 810 of process 800, and/or at 904 of process 900), another system, or the like. At 1104, in response to receiving the request, process 1100 comprises generating, by the system, the synthetic lesion images using a synthetic lesion generation model trained to generate the synthetic lesion image data objects and tailor the synthetic lesion image data objects to account for different types of lesions and different anatomical locations of the lesions (e.g., via the lesion augmentation component 102).

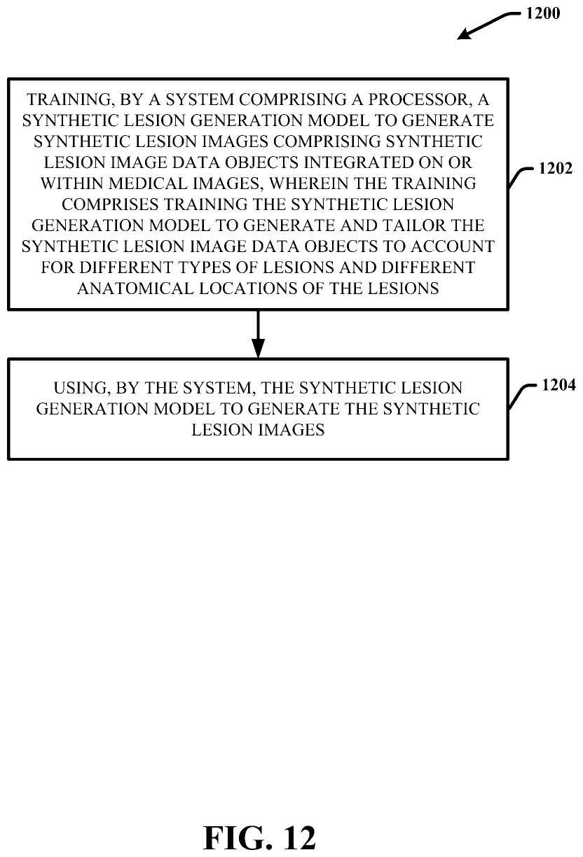

FIG. 12 illustrates a block diagram of another example, non-limiting computer implemented method 1200 for augmenting CXR images with synthetic lesions in accordance with one or more embodiments of the disclosed subject matter. Process 1200 comprises, at 1202, training, by a system comprising a process (e.g., computing system 100) a synthetic lesion generation model to generate synthetic lesion images comprising synthetic lesion image data objects integrated on or within medical images, wherein the training comprises training the synthetic lesion generation model to generate and tailor the synthetic lesion image data objects to account for different types of lesions and different anatomical locations of the lesions (e.g., via training component 108). At 1204, process 1200 comprises using the synthetic lesion generation model to generate the synthetic lesion images (e.g., via the lesion augmentation component 102).

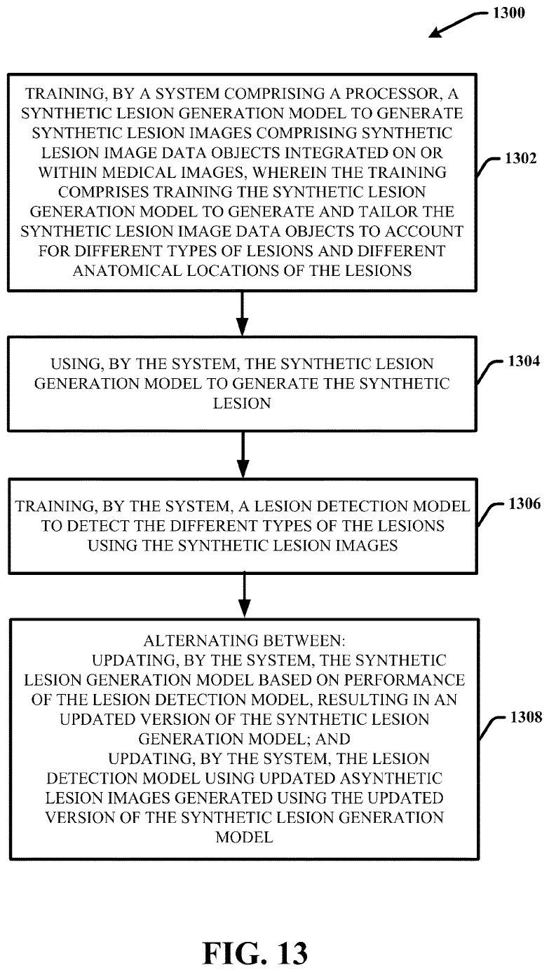

FIG. 13 illustrates a block diagram of an example, non-limiting computer implemented method 1300 for augmenting CXR images with synthetic lesions and employing the augmented images in association with optimizing a thoracic disease detection model, in accordance with one or more embodiments of the disclosed subject matter. Process 1300 comprises, at 1302, training, by a system comprising a process (e.g., computing system 100) a synthetic lesion generation model to generate synthetic lesion images comprising synthetic lesion image data objects integrated on or within medical images, wherein the training comprises training the synthetic lesion generation model to generate and tailor the synthetic lesion image data objects to account for different types of lesions and different anatomical locations of the lesions (e.g., via training component 108). At 1304, process 1300 comprises using the synthetic lesion generation model to generate the synthetic lesion images (e.g., via the lesion augmentation component 102). At 1306, process 1300 comprises training, by the system, a lesion detection model to detect the different types of the lesions using the synthetic lesion images (e.g., via training component 108). At 1308, process 1300 further comprises alternating between: updating the synthetic lesion generation model based on performance of the lesion detection model, resulting in an updated version of the synthetic lesion generation model; and updating the lesion detection model using updated synthetic lesion images generated using the updated version of the synthetic lesion generation model (e.g., via the training component 108).

### Example Operating Environments

One or more embodiments can be a system, a method, and/or a computer program product at any possible technical detail level of integration. The computer program product can include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It can be understood that each block of the flowchart illustrations and/or block diagrams, and combinations of blocks in the flowchart illustrations and/or block diagrams, can be implemented by computer readable program instructions.

These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession may, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In connection with FIG. 14, the systems and processes described below can be embodied within hardware, such as a single integrated circuit (IC) chip, multiple ICs, an application specific integrated circuit (ASIC), or the like. Further, the order in which some or all of the process blocks appear in each process should not be deemed limiting. Rather, it should be understood that some of the process blocks can be executed in a variety of orders, not all of which can be explicitly illustrated herein.

With reference to FIG. 14, an example environment 1400 for implementing various aspects of the claimed subject matter includes a computer 1402. The computer 1402 includes a processing unit 1404, a system memory 1406, a codec 1435, and a system bus 1408. The system bus 1408 couples system components including, but not limited to, the system memory 1406 to the processing unit 1404. The processing unit 1404 can be any of various available processors. Dual microprocessors and other multiprocessor architectures also can be employed as the processing unit 1404.

The system bus 1408 can be any of several types of bus structure(s) including the memory bus or memory controller, a peripheral bus or external bus, or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 13144), and Small Computer Systems Interface (SCSI).

The system memory 1406 includes volatile memory 1410 and non-volatile memory 1412, which can employ one or more of the disclosed memory architectures, in various embodiments. The basic input/output system (BIOS), containing the basic routines to transfer information between elements within the computer 1402, such as during start-up, is stored in non-volatile memory 1412. In addition, according to present innovations, codec 1435 can include at least one of an encoder or decoder, wherein the at least one of an encoder or decoder can consist of hardware, software, or a combination of hardware and software. Although codec 1435 is depicted as a separate component, codec 1435 can be contained within non-volatile memory 1412. By way of illustration, and not limitation, non-volatile memory 1412 can include read only memory (ROM), programmable ROM (PROM), electrically programmable ROM (EPROM), electrically erasable programmable ROM (EEPROM), Flash memory, 3D Flash memory, or resistive memory such as resistive random access memory (RRAM). Non-volatile memory 1412 can employ one or more of the disclosed memory devices, in at least some embodiments. Moreover, non-volatile memory 1412 can be computer memory (e.g., physically integrated with computer 1402 or a mainboard thereof), or removable memory. Examples of suitable removable memory with which disclosed embodiments can be implemented can include a secure digital (SD) card, a compact Flash (CF) card, a universal serial bus (USB) memory stick, or the like. Volatile memory 1410 includes random access memory (RAM), which acts as external cache memory, and can also employ one or more disclosed memory devices in various embodiments. By way of illustration and not limitation, RAM is available in many forms such as static RAM (SRAM), dynamic RAM (DRAM), synchronous DRAM (SDRAM), double data rate SDRAM (DDR SDRAM), and enhanced SDRAM (ESDRAM) and so forth.

Computer 1402 can also include removable/non-removable, volatile/non-volatile computer storage medium. FIG. 14 illustrates, for example, disk storage 1410. Disk storage 1410 includes, but is not limited to, devices like a magnetic disk drive, solid state disk (SSD), flash memory card, or memory stick. In addition, disk storage 1410 can include storage medium separately or in combination with other storage medium including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 1410 to the system bus 1408, a removable or non-removable interface is typically used, such as interface 1416. It is appreciated that disk storage 1410 can store information related to a user. Such information might be stored at or provided to a server or to an application running on a user device. In one embodiment, the user can be notified (e.g., by way of output device(s) 1436) of the types of information that are stored to disk storage 1410 or transmitted to the server or application. The user can be provided the opportunity to opt-in or opt-out of having such information collected or shared with the server or application (e.g., by way of input from input device(s) 1428).

It is to be appreciated that FIG. 14 describes software that acts as an intermediary between users and the basic computer resources described in the suitable operating environment 1400. Such software includes an operating system 1410. Operating system 1410, which can be stored on disk storage 1410, acts to control and allocate resources of the computer 1402. Applications 1420 take advantage of the management of resources by operating system 1410 through program modules 1424, and program data 1426, such as the boot/shutdown transaction table and the like, stored either in system memory 1406 or on disk storage 1410. It is to be appreciated that the claimed subject matter can be implemented with various operating systems or combinations of operating systems.

A user enters commands or information into the computer 1402 through input device(s) 1428. Input devices 1428 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1404 through the system bus 1408 via interface port(s) 1430. Interface port(s) 1430 include, for example, a serial port, a parallel port, a game port, and a universal serial bus (USB). Output device(s) 1436 use some of the same type of ports as input device(s) 1428. Thus, for example, a USB port can be used to provide input to computer 1402 and to output information from computer 1402 to an output device 1436. Output adapter 1434 is provided to illustrate that there are some output devices 1436 like monitors, speakers, and printers, among other output devices 1436, which require special adapters. The output adapters 1434 include, by way of illustration and not limitation, video and sound cards that provide a means of connection between the output device 1436 and the system bus 1408. It should be noted that other devices or systems of devices provide both input and output capabilities such as remote computer(s) 1438.

Computer 1402 can operate in a networked environment using logical connections to one or more remote computers, such as remote computer(s) 1438. The remote computer(s) 1438 can be a personal computer, a server, a router, a network PC, a workstation, a microprocessor based appliance, a peer device, a smart phone, a tablet, or other network node, and typically includes many of the elements described relative to computer 1402. For purposes of brevity, only a memory storage device 1440 is illustrated with remote computer(s) 1438. Remote computer(s) 1438 is logically connected to computer 1402 through a network interface 1442 and then connected via communication connection(s) 1444. Network interface 1442 encompasses wire or wireless communication networks such as local-area networks (LAN) and wide-area networks (WAN) and cellular networks. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 1444 refers to the hardware/software employed to connect the network interface 1442 to the bus 1408. While communication connection 1444 is shown for illustrative clarity inside computer 1402, it can also be external to computer 1402. The hardware/software necessary for connection to the network interface 1442 includes, for exemplary purposes only, internal and external technologies such as, modems including regular telephone grade modems, cable modems and DSL modems, ISDN adapters, and wired and wireless Ethernet cards, hubs, and routers.

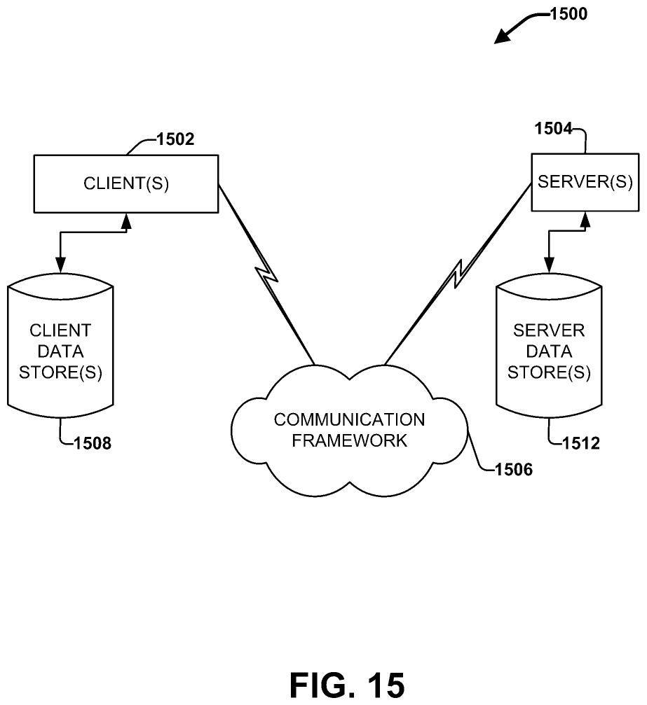

FIG. 15 is a schematic block diagram of a sample-computing environment 1500 with which the subject matter of this disclosure can interact. The system 1500 includes one or more client(s) 1502. The client(s) 1502 (e.g., corresponding to client system 700 in some embodiments) can be hardware and/or software (e.g., threads, processes, computing devices). The system 1500 also includes one or more server(s) 1504 (e.g., corresponding to vendor system 600 in some embodiments). Thus, system 1500 can correspond to a two-tier client server model or a multi-tier model (e.g., client, middle tier server, data server), amongst other models. The server(s) 1504 can also be hardware and/or software (e.g., threads, processes, computing devices). The servers 1504 can house threads to perform transformations by employing this disclosure, for example. One possible communication between a client 1502 and a server 1504 may be in the form of a data packet transmitted between two or more computer processes (e.g., process 1001 and process 1002 for example).

The system 1500 includes a communication framework 1506 that can be employed to facilitate communications between the client(s) 1502 and the server(s) 1504. The client(s) 1502 are operatively connected to one or more client data store(s) 1508 that can be employed to store information local to the client(s) 1502. Similarly, the server(s) 1504 are operatively connected to one or more server data store(s) 1512 that can be employed to store information local to the servers 1504.

It is to be noted that aspects or features of this disclosure can be exploited in substantially any wireless telecommunication or radio technology, e.g., Wi-Fi; Bluetooth; Worldwide Interoperability for Microwave Access (WiMAX); Enhanced General Packet Radio Service (Enhanced GPRS); Third Generation Partnership Project (3GPP) Long Term Evolution (LTE); Third Generation Partnership Project 2 (3GPP2) Ultra Mobile Broadband (UMB); 3GPP Universal Mobile Telecommunication System (UMTS); High Speed Packet Access (HSPA); High Speed Downlink Packet Access (HSDPA); High Speed Uplink Packet Access (HSUPA); GSM (Global System for Mobile Communications) EDGE (Enhanced Data Rates for GSM Evolution) Radio Access Network (GERAN); UMTS Terrestrial Radio Access Network (UTRAN); LTE Advanced (LTE-A); etc. Additionally, some or all of the aspects described herein can be exploited in legacy telecommunication technologies, e.g., GSM. In addition, mobile as well non-mobile networks (e.g., the Internet, data service network such as internet protocol television (IPTV), etc.) can exploit aspects or features described herein.

While the subject matter has been described above in the general context of computer-executable instructions of a computer program that runs on a computer and/or computers, those skilled in the art will recognize that this disclosure also can or may be implemented in combination with other program modules. Generally, program modules include routines, programs, components, data structures, etc. that perform particular tasks and/or implement particular abstract data types. Moreover, those skilled in the art will appreciate that the inventive methods may be practiced with other computer system configurations, including single-processor or multiprocessor computer systems, mini-computing devices, mainframe computers, as well as personal computers, hand-held computing devices (e.g., PDA, phone), microprocessor-based or programmable consumer or industrial electronics, and the like. The illustrated aspects may also be practiced in distributed computing environments where tasks are performed by remote processing devices that are linked through a communications network. However, some, if not all aspects of this disclosure can be practiced on stand-alone computers. In a distributed computing environment, program modules may be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server can be a component. One or more components may reside within a process and/or thread of execution and a component may be localized on one computer and/or distributed between two or more computers.

In another example, respective components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

Various aspects or features described herein can be implemented as a method, apparatus, system, or article of manufacture using standard programming or engineering techniques. In addition, various aspects or features disclosed in this disclosure can be realized through program modules that implement at least one or more of the methods disclosed herein, the program modules being stored in a memory and executed by at least a processor. Other combinations of hardware and software or hardware and firmware can enable or implement aspects described herein, including a disclosed method(s). The term “article of manufacture” as used herein can encompass a computer program accessible from any computer-readable device, carrier, or storage media. For example, computer readable storage media can include but are not limited to magnetic storage devices (e.g., hard disk, floppy disk, magnetic strips . . . ), optical discs (e.g., compact disc (CD), digital versatile disc (DVD), blu-ray disc (BD) . . . ), smart cards, and flash memory devices (e.g., card, stick, key drive . . . ), or the like.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor may also be implemented as a combination of computing processing units.

In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory.

By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or methods herein are intended to include, without being limited to including, these and any other suitable types of memory.

It is to be appreciated and understood that components, as described with regard to a particular system or method, can include the same or similar functionality as respective components (e.g., respectively named components or similarly named components) as described with regard to other systems or methods disclosed herein.

What has been described above includes examples of systems and methods that provide advantages of this disclosure. It is, of course, not possible to describe every conceivable combination of components or methods for purposes of describing this disclosure, but one of ordinary skill in the art may recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

What is claimed is:

1. A system, comprising:
a memory that stores computer-executable components; and
a processor that executes the computer-executable components stored in the memory, wherein the computer-executable components comprise:
a lesion augmentation component that:
generates synthetic lesion images comprising synthetic lesion image data objects integrated on or within medical images using a synthetic lesion generation model, trained to generate the synthetic lesion image data objects, and
tailor the synthetic lesion image data objects to account for different types of lesions and different anatomical locations of the lesions.

2. The system of claim 1, wherein the different types of lesions correspond to different types of thoracic diseases.

3. The system of claim 2, wherein the medical images comprise chest X-ray images.

4. The system of claim 1, wherein the lesion augmentation component adds the synthetic lesion images to a lesion image training dataset comprising lesion images, the lesion images comprising the augmented medial images, and wherein the computer-executable components further comprise:
a training component that employs the lesion image training dataset to train a lesion detection model to detect the different types of lesions in the lesion images.

5. The system of claim 4, wherein the lesion augmentation component generates the synthetic lesion images in association with reception of annotation data indicating a defined disease type, a defined anatomical location and a defined size of respective objects of the synthetic lesion image data objects for integration on or within the medical images, and
wherein the training component employs the annotation data respectively associated with the synthetic lesion images as ground truth information in association with training the lesion detection model.

6. The system of claim 4, wherein the computer-executable components further comprise:
a performance assessment component that identifies one or more target lesion images of lesion image training dataset associated with a negative performance criterion of the lesion detection model, and
wherein the training component updates the synthetic lesion generation model based on the one or more target lesion images.

7. The system of claim 1, wherein the synthetic lesion generation model is further trained to tailor the synthetic lesion data objects to account for different lesion sizes and textures.

8. The system of claim 1, wherein the computer-executable components further comprise
a training component that trains the synthetic lesion generation model using one or more machine learning processes.

9. The system of claim 8, wherein the one or more machine learning processes comprise an adversarial training process employing a lesion generator network and a discriminator network.

10. The system of claim 9, wherein the lesion generator network comprises convolutional layers and transformers.

11. The system of claim 8, wherein the synthetic lesion generation model comprises a style variation module that generates different style variations of the synthetic lesion image data objects using noise injection.

12. A method, comprising:
receiving, by a system comprising a processor, a request to generate synthetic lesion images comprising synthetic lesion image data objects integrated on or within medical images; and
in response to receiving the request, generating, by the system, the synthetic lesion images using a synthetic lesion generation model trained to generate the synthetic lesion image data objects and tailor the synthetic lesion image data objects to account for different types of lesions and different anatomical locations of the lesions.

13. The method of claim 12, wherein the different types of lesions correspond to different types of thoracic diseases, and wherein medical images comprise chest X-ray images.

14. The method of claim 12, wherein the request comprises annotation data indicating a defined disease type, a defined anatomical location and a defined size of respective objects of the synthetic lesion image data objects for integration on or within the medical images, and wherein the generating comprises generating the respective objects and integrating the respective objects on or within the medical images in accordance with the annotation data.

15. The method of claim 12, further comprising:
adding, by the system, the synthetic lesion images to a lesion image training dataset comprising lesion images, the lesion images comprising the augmented medial images; and
employing, by the system, the lesion image training dataset to train a lesion detection model to detect the different types of lesions in the lesion images.

16. The method of claim 15, wherein the generating comprises generating the synthetic lesion images in association with reception of annotation data indicating a defined disease type, a defined anatomical location and a defined size of respective objects of the synthetic lesion image data objects for integration on or within the medical images, and wherein the method further comprises:
employing, by the system, the annotation data respectively associated with the synthetic lesion images as ground truth information in association with training the lesion detection model.

17. The method of claim 15, further comprising:
identifying, by the system, one or more target lesion images of a lesion image training dataset associated with a negative performance criterion of the lesion detection model; and
updating, by the system, the synthetic lesion generation model based on the one or more target lesion images.

18. The method of claim 12, further comprising:
training, by the system, the synthetic lesion generation model using one or more machine learning processes, wherein the one or more machine learning processes comprise an adversarial training process employing a lesion generator network and a discriminator network.

19. A non-transitory machine-readable storage medium, comprising executable instructions that, when executed by a processor, facilitate performance of operations, comprising:
training a synthetic lesion generation model to generate synthetic lesion images comprising synthetic lesion image data objects integrated on or within medical images, wherein the training comprises training the synthetic lesion generation model to generate and tailor the synthetic lesion image data objects to account for different types of lesions and different anatomical locations of the lesions; and
using the synthetic lesion generation model to generate the synthetic lesion images.

20. The non-transitory machine-readable storage medium of claim 19, the operations further comprising:
training a lesion detection model to detect the different types of the lesions using the synthetic lesion images; and
alternating between:
updating the synthetic lesion generation model based on performance of the lesion detection model, resulting in an updated version of the synthetic lesion generation model; and
updating the lesion detection model using updated synthetic lesion images generated using the updated version of the synthetic lesion generation model.
